(19) United States
(12) Patent Application Publication
(10) Pub. No.: US 2009/0202801 A1
(43) Pub. Date: Aug. 13, 2009

(54) Stretched polymers, products containing stretched polymers, and their methods of manufacture and examination

(76) Inventor: John N. Magno, Middletown, NJ (US)

Correspondence Address: FOLEY AND LARDNER LLP;SUITE 500, 3000 K STREET NW, WASHINGTON, DC, 20007, US

(21) Appl. No.: 12/149,539

(22) Filed: May 2, 2008

Related U.S. Application Data
(60) Provisional application No. 60/916,066, filed on May 4, 2007.
(60) Provisional application No. 60/960,250, filed on Sep. 21, 2007.

(51) Int. Cl. B32B 7/02; D03D 25/00; D04H 13/00; D01D 5/12; C08F 10/02; C08F 10/06; G01N 33/00

(52) U.S. Cl. 428/212; 442/181; 442/327; 264/210.1; 526/348.1; 73/866

(57) ABSTRACT

Certain stretched polymers have defects which reduce their tenacity and effectively render the stretched polymers opaque. These defects are at least in part cause by the undesirable stresses being applied to the fiber. Such stresses may be avoided by avoiding thermal shock and by avoiding bending and twisting during processing. Such stretched polymers may be used in optically clear application such as bullet resistant glass.

### RELATED APPLICATIONS

This application claims priority from, and incorporates by reference, U.S. Provisional application Ser. No. 60/916,066, filed May 4, 2007, and U.S. Provisional application Ser. No. 60/960,250, filed Sep. 21, 2007.

### FIELD OF THE INVENTION

The present invention relates generally to stretched polymers, methods of making stretched polymers and methods of examining stretched polymers, and more particularly, to ultra-high molecular weight stretched polymers having new and/or improved properties, methods of making ultra-high molecular weight stretched polymers having new and/or improved properties and methods of examining stretched polymers.

### BACKGROUND

Theoretical analysis indicates stretched ultra-high molecular weight polyethylene (UHMWPE) fibers should have tenacities of 20 Gpa or greater. Yet commercially available UHMWPE fibers have only achieved tenacities of up to about a fifth of the theoretical value despite a great effort to improve tenacity over at least the last two decades. Furthermore, the opacity of commercially available UHMWPE fibers limits the number of applications in which it may be utilized. Accordingly, there is a strong need in the art to improve the tenacity of fibers and/or to have them be optically clear.

### SUMMARY OF THE INVENTION

An aspect of the present invention is to provide an (to be completed upon finalization of the claims). An aspect of the present invention is to provide a polymer element including a stretched polymer having a defect density of less than 2000 per meter of stretched polymer and a tenacity of 2 GPA. The stretched polymer may be formed from a polyolefin such as a polyethylene or a polypropylene. The stretched polymer may be formed from at least a first copolymer and a second copolymer. The refractive index of the stretch polymer may be varied according to the relative amounts of the first copolymer and the second copolymer. The stretched polymer may form one of a fiber, a yarn or a tape. The defect density may be less than 1000 per meter of stretched polymer, or more advantageously the defect density may be less than 400 per meter of stretched polymer, or more advantageously the defect density may be less than 200 per meter of stretched polymer, or more advantageously the defect density may be less than 100 per meter of stretched polymer, or more advantageously the defect density may be less than 20 per meter of stretched polymer. The stretched polymer may be transparent. When the stretched polymer is stretched such that molecules of the stretched polymer need not be not uniaxial aligned. The stretched polymer may be either woven or unwoven. When the stretched polymer is transparent, it may be contained within a transparent medium that is index matched to an index of the stretched polymer.

Another aspect of the present invention is to provide a polymer filament including a stretched polyethylene polymer having a defect density of less than 2000 per meter of stretched polymer and a tenacity of 2 GPA. The stretched polyethylene polymer may be transparent. The stretched polyethylene may be stretched such that molecules of the stretched polymer are not uniaxial aligned. The stretched polyethylene polymer may be part of a woven product, an unwoven product such as is a pre-preg product.

Another aspect of the present invention is to provide a polymer element including a stretched polymer having a tenacity greater than 7 GPa. The stretched polymer may be formed from a polyolefin such as a polyethylene or a polypropylene. The stretched polymer may be formed from at least a first copolymer and a second copolymer. The refractive index of the stretch polymer may vary according to the relative amounts of the first copolymer and the second copolymer. The stretched polymer may form one of a fiber, a yarn or a tape. The stretched polymer may have a defect density is less than 2000 per meter of stretched polymer, or more advantageously the defect density may be less than 1000 per meter of stretched polymer, or more advantageously the defect density may be less than 400 per meter of stretched polymer, or more advantageously the defect density may be less than 200 per meter of stretched polymer, or more advantageously the defect density may be less than 100 per meter of stretched polymer, or more advantageously the defect density may be less than 20 per meter of stretched polymer. The stretched polymer may be transparent. The stretched polymer may be stretched such that molecules of the stretched polymer are not uniaxial aligned. The stretched polymer may be either woven or unwoven. The stretched polymer may be transparent and contained within a transparent medium that is index matched to an index of the stretched polymer. The stretched polymer may have a tenacity greater than 10 GPa, or more advantageously the stretched polymer may have a tenacity greater than 15 GPa, or more advantageously the stretched polymer may have a tenacity greater than 18 GPa, or more advantageously the stretched polymer may have a tenacity greater than 20 GPa.

Another aspect of the present invention is to provide a polymer filament including a stretched polyethylene polymer having a tenacity greater than 7 GPa. The stretched polyethylene polymer may be transparent. The stretched polyethylene stretched such that molecules of the stretched polymer need not be uniaxial aligned. The stretched polyethylene polymer may be part of a woven product, may be part of an unwoven product. The unwoven product may be a pre-preg product.

Another aspect of the present invention is to provide a polymer element including a stretched polymer having a tenacity greater than 77 g/denier. The stretched polymer may be formed from a polyolefin such as a polyethylene or a polypropylene. The stretched polymer may be formed from at least a first copolymer and a second copolymer. The refractive index of the stretch polymer may be varied according to the relative amounts of the first copolymer and the second copolymer. The stretched polymer may form one of a fiber, a yarn or a tape. The stretched polymer may be a defect density is less than 2000 per meter of stretched polymer. The defect density may be less than 1000 per meter of stretched polymer, or more advantageously the defect density may be less than 400 per meter of stretched polymer, or more advantageously the defect density may be less than 200 per meter of stretched polymer, or more advantageously the defect density may be less than 100 per meter of stretched polymer, or more advantageously the defect density may be less than 20 per meter of stretched polymer. The stretched polymer may be transparent. The stretched polymer may be stretched such that molecules of the stretched polymer are not uniaxial aligned. The stretched polymer may be either woven or unwoven. When the stretched polymer is transparent and it may be contained within a transparent medium that is index matched to an index of the stretched polymer. The stretched polymer may have a tenacity greater than 90 g/denier, or more advantageously the stretched polymer may have a tenacity greater than 100 g/denier, or more advantageously the stretched polymer may have a tenacity greater than 110 g/denier, or more advantageously the stretched polymer has a tenacity greater than 120 g/denier.

Another aspect of the present invention is to provide a polymer filament including a stretched polyethylene polymer having a tenacity greater than 77 g/denier. The stretched polyethylene polymer may be transparent. The stretched polyethylene may be stretched such that molecules of the stretched polymer are not uniaxial aligned. The stretched polymer may be either woven or unwoven. When the stretched polymer is transparent and it may be contained within a transparent medium that is index matched to an index of the stretched polymer.

Another aspect of the present invention is to provide a polymer filament including a stretched polyethylene polymer having a high average molecular weight and a tenacity greater than 77 g/denier.

Another aspect of the present invention is to provide a polymer filament including a stretched polyethylene polymer having a stretch ratio in excess of 20 and has a ratio of tenacity to stretch ratio (t/Rₛ) of greater than 0.16 GPa.

Another aspect of the present invention is to provide a polymer filament including a stretched polyethylene polymer having a stretch ratio in excess of 40 and has a ratio of tenacity to stretch ratio (t/Rₛ) of greater than 0.10 GPa. The stretched polymer may be a stretched polyethylene polymer.

Another aspect of the present invention is to provide a polymer filament including a stretched polymer having a stretch ratio in excess of 60 and has a ratio of tenacity to stretch ratio (t/Rₛ) of greater than 0.07 GPa. The stretched polymer may be a stretched polyethylene polymer.

Another aspect of the present invention is to provide a polymer filament including a stretched polymer where (t/log₁₀Rₛ)≧32−0.095Rₛ, where t is tenacity in grams/denier and Rₛ is the stretch ratio. The stretched polymer may be a stretched polyethylene polymer.

Another aspect of the present invention is to provide a method of testing stretched polymers including placing a stretched polymer have a refractive index in a fluid having an index of refraction within 0.1 of the refractive index of the stretched polymer, and observing the stretched polymer with a polarizing microscope in the dark field mode after at least a predetermined amount of time. The stretched polymer may be a stretched polyethylene polymer.

Another aspect of the present invention is to provide a method of manufacturing stretched polymers including providing raw materials, mixing the raw materials, heating the raw materials such that the raw materials become a slurry, extruding and then cooling the slurry to form xerogel, and stretching the xerogel to form a stretch polymer. The mixing the raw materials may include a first mixing and a second mixing. The raw materials may include mineral oil and wherein the heating the raw materials occurs for a period of time insufficient to substantially degrade the mineral oil. The cooling the xerogel may be performed in a plurality of stages. Each successive stage of the plurality of stages may have a temperature lower than a preceding stage of the plurality of stages. The cooling the xerogel may be performed in a single stage having a graduated temperature from an entry point into the single stage to an exit point of the single stage. The method may further include degassing the raw materials.

Another aspect of the present invention is to provide a method of manufacturing stretched polymers including extruding and then cooling a slurry of polyethylene and mineral oil to form a gel, removing the mineral oil from the gel to form unstretched polymer, and at least partially stretching the unstretched polymer substantially without applying non-axial stresses. The non-axial stresses may include bending the unstretched polymer. Removing the mineral oil from the gel to form unstretched polymer may occur substantially without applying non-axial stresses. The non-axial stresses may include bending either the gel or the unstretched polymer.

### BRIEF DESCRIPTION OF THE DRAWINGS

The invention will be described in detail with reference to the following drawings in which like reference numerals refer to like elements wherein:

FIG. 1 is a conventional photograph of a conventional stretched UHMWPE fiber observed under a Niemarski microscope in the dark field mode with under magnification;

FIG. 2 is another photograph of a conventional stretched UHMWPE fiber observed under a Niemarski microscope in the dark field mode with under magnifications without any index matching fluid;

FIG. 3 is a photograph of a prior-art UHMWPE fiber immersed an index matching fluid and viewed under magnification with a polarizing microscope;

FIG. 4 is a photograph of a prior-art UHMWPE fiber placed under tension, immersed an index matching fluid and viewed under magnification with a polarizing microscope;

FIG. 5 is a photograph of prior art fiber and a fiber prepared according to example 1;

FIG. 6 illustrates a fiber fabrication machine 600 that may be used for producing fiber;

FIG. 7 illustrates a fiber fabrication machine 700 that may be used for producing fiber;

FIG. 8 shows a section of good quality fiber under magnification that has had the mineral oil removed but has not been stretched;

FIG. 9 shows the section of good quality fiber under magnification of FIG. 8 after bending it around a small radius curve with little or no tension;

FIG. 10 shows an unstretched fiber section having defects under magnification;

FIG. 11 shows a stretched fiber section having defects under magnification; and

FIG. 12 illustrates a cross section of a pre-preg product.

### DETAILED DESCRIPTION

Commercially available stretched ultra-high molecular weight polyethylene (UHMWPE) fibers only have tenacities of less than about a fifth of their predicted theoretical values whereas fibers made from other materials achieve far greater percentages of their theoretical tenacities. Because of this, a great deal of analysis and research has been done over the last two decades on stretched UHMWPE fibers to understand and improve its tenacity. Unfortunately, all of this analysis and research has yielded only small incremental increases in the tenacity of stretched UHMWPE fibers.

The reason conventional stretched UHMWPE fibers do not achieve higher tenacities is that the fibers have a large number of previously unidentified defects. These defects become visible upon illumination to a polarizing microscope such as Niemarski in the dark field mode under magnification once the fibers have been immersed in index matching fluid with a refractive index between 1.45 and 1.52 for a few minutes. These defects appear to be cracks, fractures, crystal dislocations or some other manifestation of a problem. Some of these defects are clearly occurring generally periodically along the fiber and have a generally similar appearance while the remaining defects seem to occur at random intervals and have an appearance that differs from each other and that differs from the defects are clearly occurring periodically.

The failure to identify these previously unknown defects likely result from a number of factors. First, standard scanning electron microscopes needs a conductive layer be deposited on the fiber before analysis because the fiber is a poor conductor. This may have masked the presence of the defects such as the defects that appear under tension. Second, conventional stretched UHMWPE fiber was believed to be opaque. This belief may have resulted from a number of things such as the somewhat rough surface of conventional stretched UHMWPE fibers, the defects acting as scattering centers, processing in oxygen which may have increased the absorption of light in the fiber, processing with over heated mineral oil that could increased the absorption of light in the fiber, and the delay before index matching fluids provide a viewable result. Third, there are a large number of factors which affect the tenacity of a fiber. The factors may have diverted researches efforts away from the real reason for the low tenacity of the fibers.

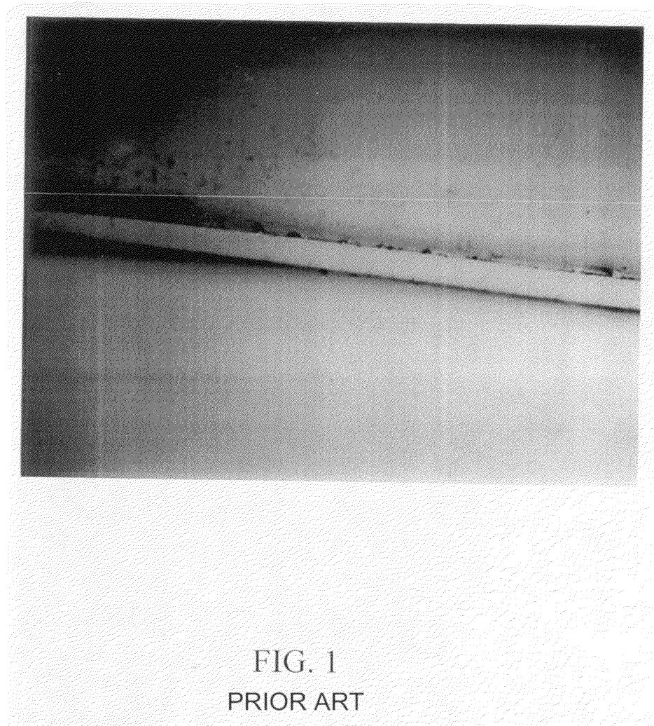

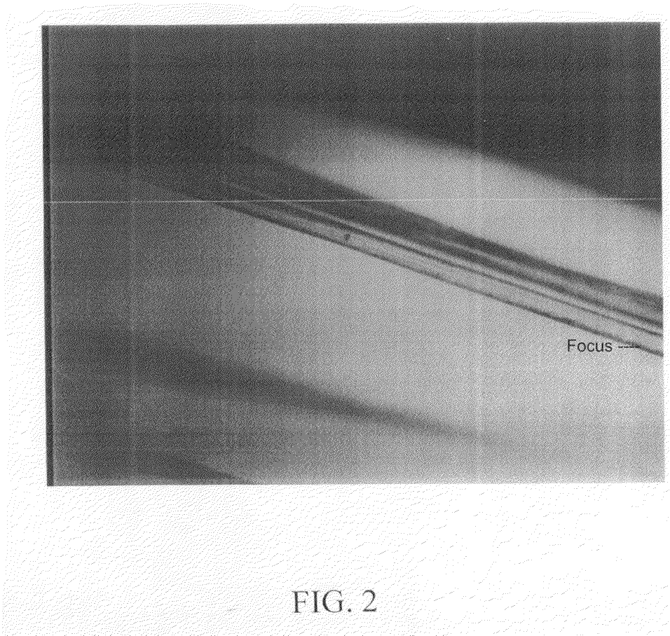

FIG. 1 is a conventional photograph of a conventional stretched UHMWPE fiber observed under a Niemarski microscope in the dark field mode with under magnification. The conventional stretched UHMWPE fiber appears to be structurally sound. Similarly, FIG. 2 is another photograph of a conventional stretched UHMWPE fiber observed under a Niemarski microscope in the dark field mode with under magnifications without any index matching fluid. As can be seen in FIG. 2, there are no obvious defects. Upon the addition of index matching fluid and waiting a short period of time, defects in the fiber will become obvious.

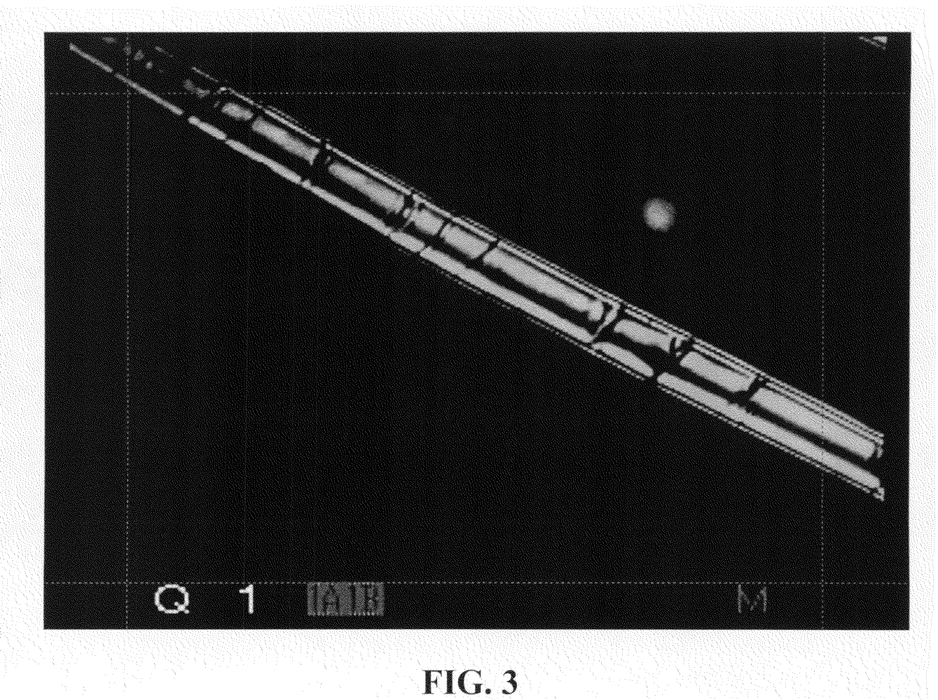

FIG. 3 is a photograph of a prior-art UHMWPE fiber immersed an index matching fluid and viewed under magnification with a polarizing microscope. A series of defects are seen to cross the fiber at an angle roughly perpendicular to its long axis. The density of the defects in this fiber is on the order of 200 defects per millimeter. In general the defect densities of prior art UHMWPE fibers is in the range of about 200 to about 900 or more defects per millimeter. The exact density and severity of the defects appears to vary somewhat on a fiber to fiber basis.

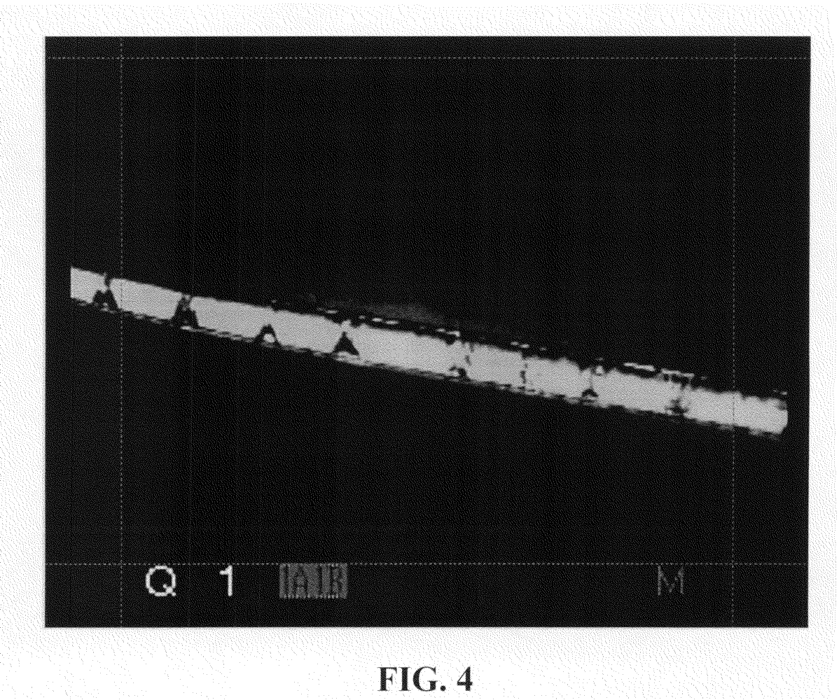

FIG. 4 is a photograph of a prior-art UHMWPE fiber placed under tension, immersed an index matching fluid and viewed under magnification with a polarizing microscope. The tension was created by adhering cellophane tape to the two opposite ends of a segment of prior-art UHMWPE fiber and manually pulling the tape. The tension opens up the defects into wider wedge-shaped cracks. The fiber appears to be on its way to failing under this stress, which suggests that these defects to be a cause for the failure of stretched UHMWPE fibers at a level of tension that is lower than that predicted theoretically. Specifically, the defects shown in FIG. 4 strongly suggest that at least part of the fiber is compromised due to these defects. Since it appears that all prior-art UHMWPE fibers have such defects while nonetheless achieving about a fifth of their theoretical strength, it is believed that such defects are primarily a surface phenomenon that leaves a central core of the fiber intact. This would help explain why UHMWPE fibers have achieved only a fifth of their theoretical strength.

The tenacity of UHMWPE fibers may be increased by reducing the frequency and/or severity of the defects. It is believed that the defects result from undesirable stress effects during the manufacturing process. These undesirable stress effects may be created in several ways. One way undesirable stress effects may occur is the quenching of the fiber may be occurring so rapidly that thermal shock is causing defects. It is believed that thermal shock is resulting in the defects that occur at random intervals. Thus, if the fiber is quenched and/or cooled so as to avoid thermal shock, any defects caused by thermal shock will be reduced or eliminated. For example, a multistage quenching bath or a bath having a gradation of it temperature may be used to reduce or eliminate thermal shock. Another way undesirable stress effects occur is through undesirable mechanical stresses being applied while the fiber is at an elevated temperature. It is believed that at an elevated temperature, any stress that is applied that is not uniform and not along the stretching direction of the fiber may result in defects. For example, twisting of the fiber may result in torsion stresses and bending of the fiber around a godet or the like may result in axial stresses, both of which result in defects. As used herein, non-axial stresses are any stresses not along the stretching axis of the polymer. Thus, by using larger godets and the like and/or stretching fibers in a straight line without any bending, the undesirable mechanical stresses may be reduced or eliminated which will also reduce the occurrence of defects.

### Example 1

A stainless steel 2.5 gallon jacketed vessel with a paddle-type stirrer was charged, in order, with a mixture of mineral oil (94.2 wt. %), a linear UHMWPE powder (5 wt. %), antioxidant powder (0.5 wt. %) and a lubricity additive (0.3 wt. %). The mineral oil used was white mineral oil. The linear UHMWPE powder was Himont UHMW 1900, the antioxidant powder was Ciba Irganox® B-225 and the lubricity additive was aluminum stearate.

This mixture was then heated at 1 deg./min. to about 150° C. with constant stirring at 10 rpm and a nitrogen blanket of 2 psi was applied to the top of the vessel for 15 hours. These parameters were maintained and created a slurry. This slurry was then left to cool to 70° C. and then transferred into a heated helical mixer preheated to 70° C.

Nitrogen was then applied to the helical mixer at 2 psi along with a motor rotation (mixing) of 5 rpm. The temperature was raised to about 155° C. at 2 deg./minute and held at about 155° C. for 30 minutes as the motor rotation was increased to 10 rpm. Next, the temperature was increased at a rate of 2° C./minute to about 180° C. and then maintained at about 180° C. for 30 minutes.

The motor rotation was then increased to 15 rpm as the nitrogen pressure was increased to 12 psi and the valve at the base of the mixer was then opened to allow flow of the slurry to the three-holed spinneret. The spinneret temperature was maintained at about 168° C. as the material flowing from the spinneret was quenched in a liquid bath of water located 6 inches below the output face of the spinneret. The spinneret hole dimensions were 0.65 mm in diameter by ¾ inch in depth.

The water bath used was a stainless steel rectangle container with dimensions of two foot wide by two foot deep by four feet long. This bath had a continuous water flow heated to a temperature of about 15° C.

The xerogel extruded uniform solution filament was then pulled down to the water and held just below the surface using 2 four-inch diameter Teflon coated rollers 3 feet apart and that spin freely and through the water at a rate of four meters a minute onto a single four-inch diameter by 6 inch long plastic spool. Once 100 meters of fiber was wound upon this single four-inch spool, the fiber was collected and the run ended.

The fiber was then immersed in xylene for 24 hours for cleaning. The fiber was then re-spooled and again immersed in xylene for another 24-hour period. This process was repeated a third time. No unnecessary tension was applied to the xerogel during the re-spooling process. Next, a heat gun set to a low temperature was used during the drying cycle by re-spooling the xerogel and using the heat gun airflow to dry the fiber, this process was repeated five times until xerogel was completely dry.

Next an 8 inch diameter by 11 inch long godet that was not motorized and an 8 inch by 11 inch long motorized godet at a 30:1 stretch ratio. Each godet worked in tandem with an air-assisted fiber idler roller. Between the two godets was a ¾ inch, six-foot long hollow copper convection-type heating tube that housed three thermocouples. The distance from the end of the heating tube to each godet was 18 inches. The heating tube also had a nitrogen purge of 2 psi going into the entry point of the fiber. The fiber were wrapped multiple times around the first idler roll and the unmotorized godet providing a non-rotational anchor point before entering the six foot copper tube. The fiber was then wrapped multiple times around the second godet and idler roll to provide another fixed anchor point. The godet was then rotated slowly to stretch to the fiber to a ratio of 30:1. This process was repeated a second time at a fiber draw ratio of 15:1 and then a third time at 15:1 for the final process. This results in a total draw ratio of about 60:1.

The resultant fibers were tested for tenacity. They had 58 to 78 g/denier as measured by ASTM D2256-2. The fibers were placed in an index matching fluid and observed with a polarizing microscope in the dark field mode. The numerous defects observed in prior art fibers were not observed in the inventive fibers.

### Example 2

An 8CV Helicone mixer with 2 helical blades was charged with about half of the mineral oil, followed by the linear UHMWPE powder, and then the rest of the mineral oil. In total, 5930 g (94.8 wt. %) of mineral oil was used and 325 g (5.2 wt. %) of linear UHMWPE powder was used. The mineral oil used was Kaydol white mineral oil from Brenntag and the linear UHMWPE powder was GUR 4120 (linear polyolefin resin in powder form with a molecular weight of ca. 5.0 mM g.mol that includes calcium sterate in a concentration of 500 part per million or 0.05% wt. % of the GUR 4120) from Ticona. The mixture is stirred at room temperature for approximately one hour.

The mixture was then heated to about 188° C. with constant stirring in the reverse direction (to facilitate upward migration of bubbles) under a full vacuum. This continued until the mixture bubbles disappeared such that the mixture was completely degassed. Once the bubbles disappeared and the mixture appeared clear, the temperature of the mixture was lowered to approximately 130° C. and a blanket of argon at 1-3 psi was applied.

Once the mixture reached approximately 130° C., the mixing speed was reduced to the slowest setting and the mixing direction was changed to the forward direction, creating a downward flow to facilitate pump feeding. The valve at the base of the mixer was then opened to allow flow of the mixture to the metering pump. The metering pump and all components in the column were maintained at approximately 130° C. except for the spinneret die which was maintained at a temperature of approximately 135° C. to approximately 140° C. There was a single 0.05 mm diameter hole in the spinneret die.

The hole in the spinneret die was submerged in a water bath. The water bath was heated to approximately 93° C. adjacent the spinneret die and cooled with ice on the opposite end creating a thermal gradient over the 14 foot bath.

The mixture was pumped through the spinneret die so as to form a gel filament. The gel filament was then pulled through the water, held just below the surface, and taken up on a 3 inch diameter core. The gel filament was rewound with no overlapping filaments and developed in a Soxlet extractor using hexane as a solvent for three complete cycles that took about a half hour each. After the third cycle, the developed fiber was air dried.

Lastly, sample pieces of developed fiber were drawn by attaching a weight to one end and suspending in a 6 foot long tube containing distilled water, uniformly heated to 180° C.

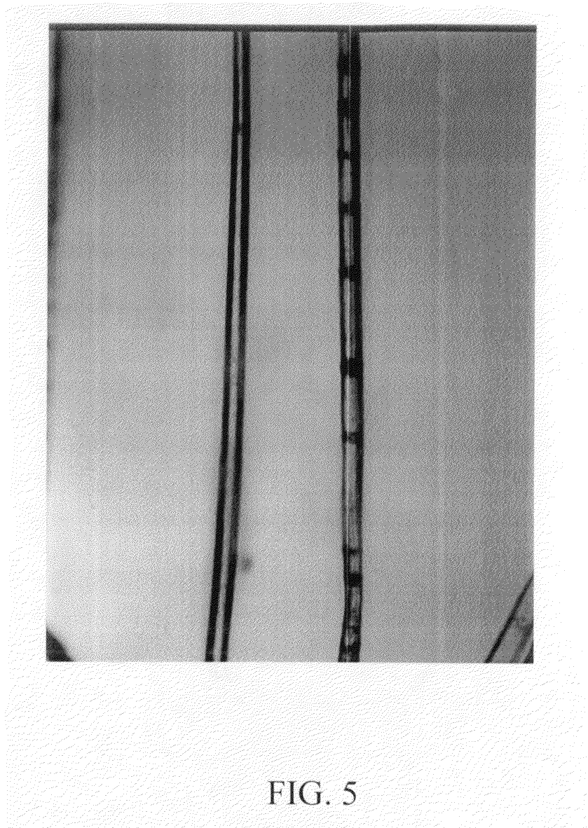

FIG. 5 is a photograph of prior art fiber and a fiber prepared according to example 1. The prior art fiber located to the right side shows numerous dark spots which are the defects. The fiber prepared according to example 1 is on the left and does not have these dark spots.

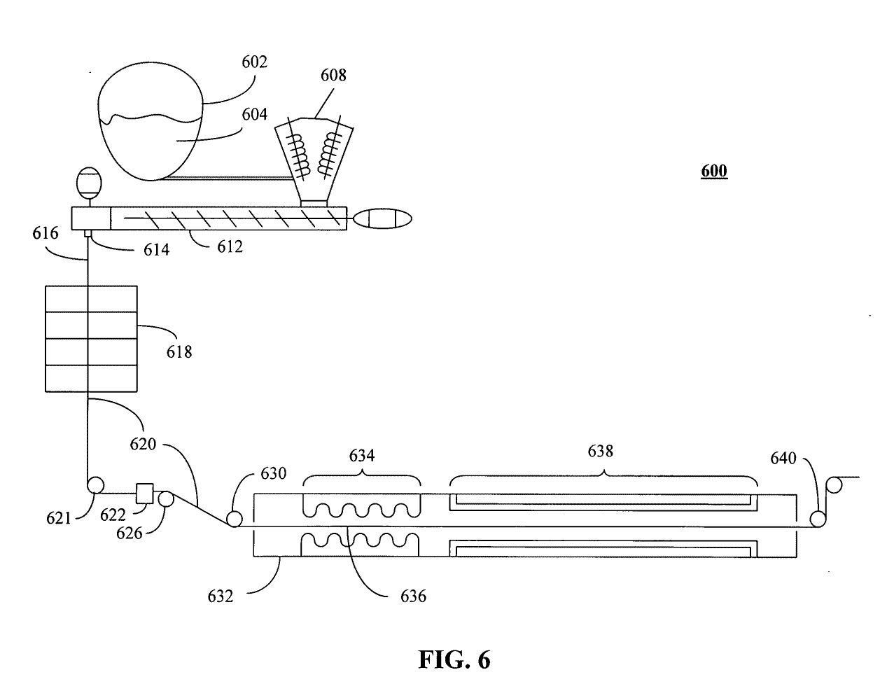

FIG. 6 illustrates a fiber fabrication machine 600 that may be used for producing fiber. The fiber fabrication machine 600 includes a premix container 602 is filled with the raw materials 602 (See Table 1 for a list of some exemplary materials) used to make the fiber. The mixed and degassed raw material 602 may be transferred into an optional storage tank (not shown) or may be directly transferred into a helical mixer 608. The mixed and degassed raw material 602 that is transferred into the helical mixer 608 is heated to above the gelation temperature of the UHMWPE powder. Some or all of the heating may occur during the transfer to the helical mixer 608 or all of the heating may occur in the helical mixer 608. The size of the helical mixer 608 is selected so as to keep a minimum the raw materials are heated to just below their liquid point so as to minimize degradation of the mineral oil. An inert gas may be added to the helical mixer 608. The inert gas prevents exposure of the raw material 602 in the helical mixer 608 to oxygen or other gases which may degrade the optical clarity of the resultant fiber. A metering pump 612 then pumps out the raw material 802 from the helical mixer 608 at the desire rate and forces it through the spinneret 614.

The spinneret 614 then extrudes one or more gel fibers 616 (the number of apertures is often between 16-240) into a cooling system 618. The cooling system 618 may be made of a plurality of baths (the baths may be all be same or may be different baths), may be a single bath having a temperature gradation, or may be a combination of both. The use of a plurality of baths allow for improved control over loading factors and all for the use of different solvents. The cooling system 618 may be a horizontal system or a vertical system. The horizontal system is advantageous in that it is closer to a conventional bath but a vertical would align gravity with the stretching direction of the one or more gel fibers 616 and might reduce the amount of mechanically induced surface features thereby making such a fiber more suitable for optically clear applications. The one or more gel fibers 616 are still able to chemically combine with oxygen or other gases which may degrade the optical clarity of the resultant fiber, so inert gas is used to fill any “air” gaps. Upon cooling down, the gel fibers 616 become stable unstretched fibers 620 that may be processed (e.g., bent, twisted, separated etc.) without creating defects. But, once the mineral oil is removed from unstretched fibers 620, the unstretched fibers 620 lose their stability and become susceptible to the creation of defects. The one or more gel fibers 616 and unstretched fibers 620 are drawn by an initial godet 621. Heating the godet 621 may reduce the number of defects.

After leaving the cooling system 618, the unstretched fibers 620 are maintained in an inert gas environment and separated from each other by a fiber separator 622. The fiber separator 622 may include a comb like structure that guides the unstretched fibers 620 onto one or more grooved drums 626. The separation of the unstretched fibers 620 helps ensure uniform stretching and orientation of molecules of the unstretched fibers 620 by a stretching system.

The separated unstretched fibers 620 are fed into stretching system. The stretching system linearly stretches the separated unstretched fibers 620 so as to minimize or eliminate the undesirable mechanical stresses that can cause defects. The first part of the stretching system is a first motorized godet 630. The first motorized godet 630 essentially takes in the separated unstretched fibers 620 with a minimal amount of tension being transferred backwards through the fiber fabrication machine 600. The first motorized godet 630 also secures the separated unstretched fibers 620 such that tension may be applied by a second motorized godet 640 which runs at a speed that is greater than the first motorized godet 630 to stretch the separated unstretched fibers 620.

From the first motorized godet 630, the separated unstretched fibers 620 are feed into a temperature controlled tube 632 that is filled with an inert gas. The temperature controlled tube 632 includes a heating portion 634 that heats the separated unstretched fibers 620 into stretchable fibers 636 that will elongate and orient under the tension created by the first and second godets 630, 640. The next part of the temperature controlled tube 632 is a cooling portion 638 that slowly cools the stretchable fibers 636 into high strength stretched UHMWPE fibers 642. The heating portion 634 is much shorter than the cooling portion 638. For example, the heating portion 634 may be 10 feet in length while the cooling portion 638 may be 90 feet in length. The stretching of the stretchable fibers 636 primarily occurs in the heating portion 634 of the temperature controlled tube 632. As the temperature of the stretchable fibers 636 cools, it becomes less stretchable and more prone to forming defects. The stretching system may be oriented may be oriented horizontally, vertically or at some angle. Additional stretching systems may be included to improve the control over the stretching process and/or to make the overall system smaller and/or to better avoid undesirable mechanical stresses.

Alternatively, space may be saved by including rollers and the like in the fiber fabrication machine 600. Any such rollers should have large radii and be located where the temperature is elevated to minimize the formation of defects. For example, rollers with radii of at least five centimetres may be used to reduce defects as compared to rollers having conventional radii. Such defects may be further reduced by using rollers with radii of at least ten centimetres and may be still further reduced by using rollers with radii of at least twenty centimetres.

Another alternative is that the cooling system 618 may use gases or spray instead of baths. Yet another alternative is to use some combination or combinations of gases, sprays and baths.

In addition to the use the invention in with respect to UHMWPE fibers, the inventive concepts disclosed herein may be used with respect to other polyolefin fibers.

The fibers produced according to the methods disclosed herein have far fewer defects than convention fibers. This allows the fibers to be used in optically clear applications such as cockpit canopies, bullet resistant windows, ultra-strong clear fishing line, ultra-strong clear coverings, clear face shields or face masks for blast protection, improved safety glass, clear hand held safety shields such as used by riot police, clear protective coverings, clear tapes, and many other applications. In military applications, clear shields could be attached to various types of carried and mounted weapon systems to reduce to stop sniper or enemy fire, or to stop shrapnel from explosions such as IEDs or rocket blasts.

The stretching ratio of the fibers may be reduced in order to improve the optical clarity of optically clear fibers. For example, the stretching ration may be reduced from that where optical clarity is irrelevant (e.g., opaque applications) by 20 to 80%. Such reductions in the stretching ratio to increase the optical clarity must be balanced against the lost tenacity of the resultant fibers.

The refractive index of the fiber may be adjusted by the substitution of some of the hydrogen atoms on the polyethylene backbone with fluorine atoms. Such a substitution may be achieved by using a polyethylene/polyvinyl fluoride copolymer as a component of or all of the material from which the stretched filaments are produced. Alternatively, the substitution may be achieved by blending polyvinyl fluoride into the polyethylene or other polyolefin from which the stretched fibers are produced. Other copolymers may also be used.

Although various aspects of the invention are discussed in terms of fibers, it is also applicable to tapes and other geometries.

Additional elements, systems and the like that are used in conventional fiber fabrication machines may be added to the fiber fabrication machine of the present invention. For example, a drying device may be included to dry the fiber. Keeping with the principles discussed above, such a drying device might blow a heated nitrogen gas instead of heated air as is conventional.

TABLE 1
Exemplary Raw Materials 602

| | |
|---|---|
| Mineral oil | Witco's Kaydol ® white mineral oil |
| UHMWPE powder (1-11 | Himont UHMW 1900, Ticona GUR 4120, |
| million molecular | Ticona GUR 4150, Ticona GUR 4170 |
| weight linear | |
| polyethylenes) | |
| Antioxidants | Ciba Irganox ® B-225 |
| Lubricity additives | Aluminum stearate, calcium sterate |
| Solvents | Tetrafluoroethane, CTFE - Genesolv, |
| | Genetron 134a, or HFC-134a, xylene, |
| | hexane |
| Inert gas | Nitrogen, Argon |

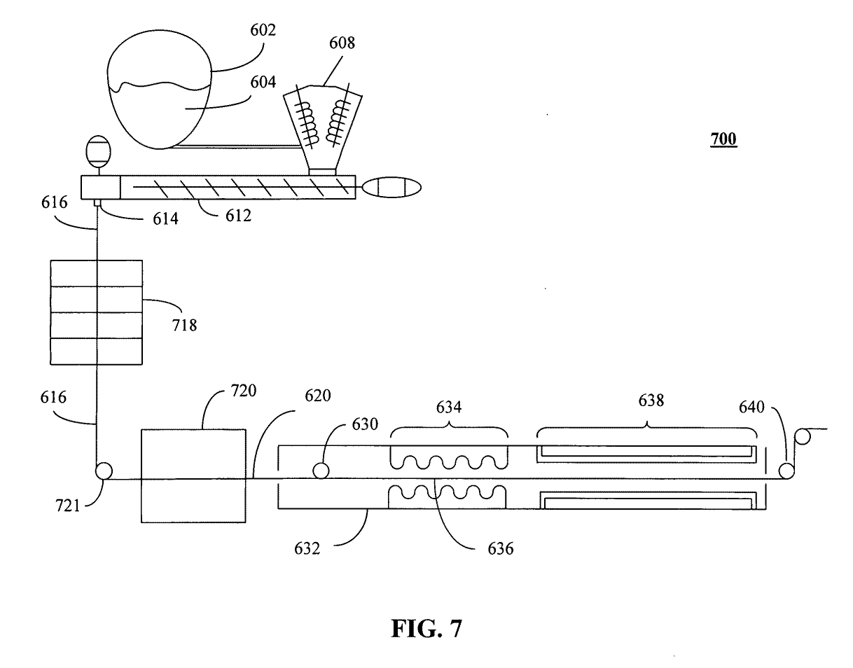

FIG. 7 illustrates a fiber fabrication machine 700 that may be used for producing fiber. The fiber fabrication machine 700 is similar to the fiber fabrication machine 600 of FIG. 6 except that the one or more gel fibers 616 are aligned along an axis prior to the removal of the mineral oil such that the undesirable defects are further reduced. The one or more gel fibers 616 are drawn through a graduated quenching bath 718 by an initial godet 721. Subsequent to the graduated quenching bath 718 and the initial godet 721 is an oil removal bath 720. The functions of cooling system 618 of FIG. 6 are separated provided by the graduated quenching bath 718 and the oil removal bath 721. The initial godet 721 is located before the oil removal bath 720 because stresses on the one or more gel fibers 616 are much less likely or do not result in the generation of defects. The placement before the oil removal bath 720 obviates the advantage of heating the initial godet 721 to further reduce the generation of defects. In order to avoid transmitting tension to the unstretched fibers 620 between the initial godet 721 and the first godet 630 in the temperature controlled tube 632, the unstretched fibers 620 may be wrapped multiple times around the first godet 630. The diameter of the first godet 630 should be made as large as practical to minimize or eliminate non-axial stresses that would otherwise generate defects.

The fiber fabrication machine 700 of FIG. 7 also differs from the fiber fabrication machine 600 of FIG. 6 in that the fiber separator 622 and the one or more grooved drums 626 are omitted as they may be more likely to increase the number of defects rather than reduce them.

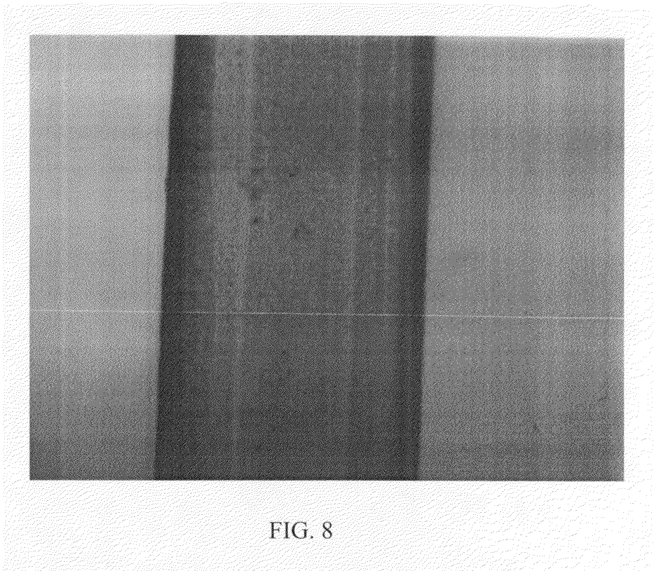

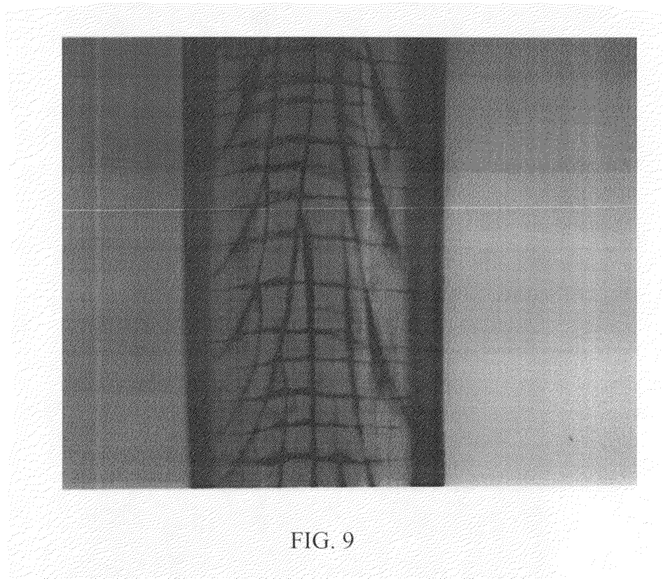

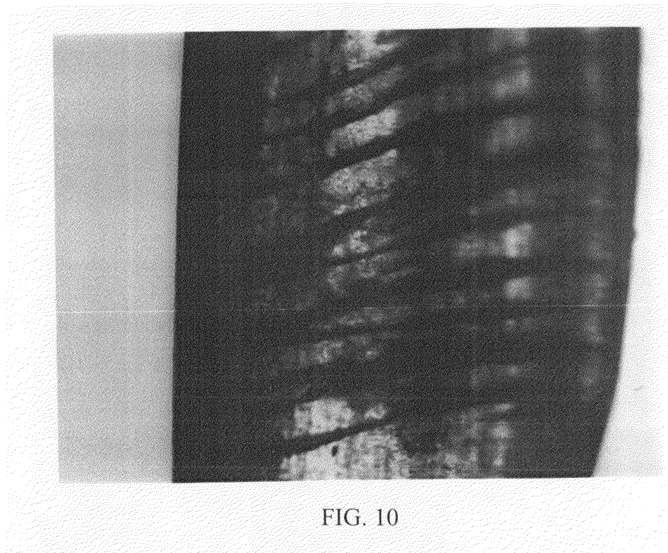

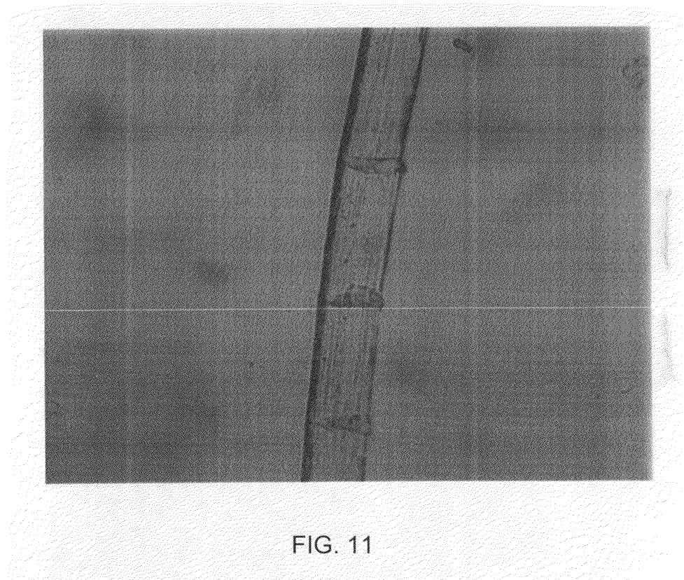

The cause or causes of fiber defects are not well understood. But it is clear that that good quality fiber when processed such that non-linear forces such as those that occur while going around a curve results in defects. For example, FIG. 8 shows a section of good quality fiber under magnification that has had the mineral oil removed but has not been stretched. Notice the absence of dark areas which are defects. Taking the good quality fiber of FIG. 8 and simply bending it around a small radius curve with little or no tension produces lots of defects (the dark areas) as is shown in FIG. 9. FIG. 10 shows another piece of unstretched fiber section having defects. Defective unstretched fiber such as in FIG. 10 becomes defective stretched fiber such as shown in FIG. 11.

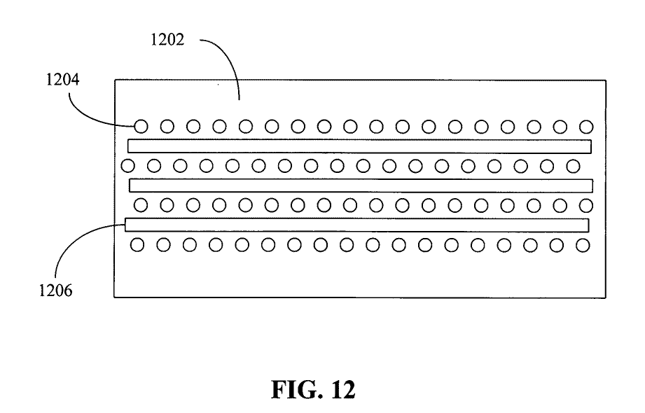

FIG. 12 illustrates a cross section of a pre-preg product. The pre-preg product includes a polymer matrix 1202 (or any other suitable material) and layers of fibers. Some of the fibers 1204 are aligned normal to the plane of the cross section while other fibers 1206 are aligned parallel to the plane of the cross section. For an optically clear or transmissive pre-preg product, the polymer matrix is index matched to the fibers 1204, 1206. Transmissive pre-preg products may be used as bullet resistant glass, safety glass, and the like. Thin layers of a transmissive pre-preg product may be used to retro-fit glass windows or the like.

Although several embodiments of the present invention and its advantages have been described in detail, it should be understood that changes, substitutions, transformations, modifications, variations, permutations and alterations may be made therein without departing from the teachings of the present invention, the spirit and the scope of the invention being set forth by the appended claims.

What is claimed is:

1. A polymer element comprising:
a stretched polymer having a defect density of less than 2000 per meter of stretched polymer and a tenacity of 2 GPA.

2. The element of claim 1, wherein the stretched polymer is formed from a polyolefin.

3. The element of claim 2, wherein the polyolefin is a polyethylene.

4. The element of claim 2, wherein the polyolefin is a polypropylene.

5. The element of claim 1, wherein the stretched polymer forms one of a fiber, a yarn or a tape.

6. The element of claim 1, wherein the defect density is less than 1000 per meter of stretched polymer.

7. The element of claim 1, wherein the defect density is less than 400 per meter of stretched polymer.

8. The element of claim 1, wherein the defect density is less than 200 per meter of stretched polymer.

9. The element of claim 1, wherein the defect density is less than 100 per meter of stretched polymer.

10. The element of claim 1, wherein the defect density is less than 20 per meter of stretched polymer.

11. The element of claim 1, wherein the stretched polymer is transparent.

12. The element of claim 11, wherein the stretched polymer is stretched such that molecules of the stretched polymer are not uniaxial aligned.

13. The element of claim 1, wherein the stretched polymer is either woven or unwoven.

14. The element of claim 1, wherein the stretched polymer is transparent and contained within a transparent medium that is index matched to an index of the stretched polymer.

15. A polymer filament comprising:
a stretched polyethylene polymer having a defect density of less than 2000 per meter of stretched polymer and a tenacity of 2 GPA.

16. The filament of claim 15, wherein the stretched polyethylene polymer is transparent.

17. The filament of claim 15, wherein the stretched polyethylene is stretched such that molecules of the stretched polymer are not uniaxial aligned.

18. The filament of claim 15, wherein the stretched polyethylene polymer is part of a woven product.

19. The filament of claim 15, wherein the stretched polyethylene polymer is part of an unwoven product.

20. The filament of claim 19, wherein the unwoven product is a pre-preg product.

21. A polymer element comprising:
a stretched polymer having a tenacity greater than 7 GPa.

22. The element of claim 21, wherein the stretched polymer is formed from a polyolefin.

23. The element of claim 22, wherein the polyolefin is a polyethylene.

24. The element of claim 22, wherein the polyolefin is a polyethylene or a polypropylene.

25. The element of claim 21, wherein the stretched polymer forms one of a fiber, a yarn or a tape.

26. The element of claim 21, wherein the stretched polymer has a defect density is less than 2000 per meter of stretched polymer.

27. The element of claim 21, wherein the stretched polymer has a defect density is less than 1000 per meter of stretched polymer.

28. The element of claim 21, wherein the stretched polymer has a defect density is less than 400 per meter of stretched polymer.

29. The element of claim 21, wherein the stretched polymer has a defect density is less than 200 per meter of stretched polymer.

30. The element of claim 21, wherein the stretched polymer has a defect density is less than 100 per meter of stretched polymer.

31. The element of claim 21, wherein the stretched polymer has a defect density is less than 20 per meter of stretched polymer.

32. The element of claim 21, wherein the stretched polymer is transparent.

33. The element of claim 32, wherein the stretched polymer is stretched such that molecules of the stretched polymer are not uniaxial aligned.

34. The element of claim 21, wherein the stretched polymer is either woven or unwoven.

35. The element of claim 21, wherein the stretched polymer is transparent and contained within a transparent medium that is index matched to an index of the stretched polymer.

36. The element of claim 21, wherein the stretched polymer has a tenacity greater than 10 GPa.

37. The element of claim 21, wherein the stretched polymer has a tenacity greater than 15 GPa.

38. The element of claim 21, wherein the stretched polymer has a tenacity greater than 18 GPa.

39. The element of claim 21, wherein the stretched polymer has a tenacity greater than 20 GPa.

40. A polymer filament comprising:
a stretched polyethylene polymer having a tenacity greater than 7 GPa.

41. The filament of claim 40, wherein the stretched polyethylene polymer is transparent.

42. The filament of claim 40, wherein the stretched polyethylene is stretched such that molecules of the stretched polymer are not uniaxial aligned.

43. The filament of claim 40, wherein the stretched polyethylene polymer is part of a woven product.

44. The filament of claim 40, wherein the stretched polyethylene polymer is part of an unwoven product.

45. The filament of claim 44, wherein the unwoven product is a pre-preg product.

46. A polymer element comprising:
a stretched polymer having a tenacity greater than 77 g/denier.

47. The element of claim 46, wherein the stretched polymer is formed from a polyolefin.

48. The element of claim 47, wherein the polyolefin is a polyethylene.

49. The element of claim 47, wherein the polyolefin is a polyethylene or a polypropylene.

50. The element of claim 46, wherein the stretched polymer forms one of a fiber, a yarn or a tape.

51. The element of claim 46, wherein the stretched polymer has a defect density is less than 2000 per meter of stretched polymer.

52. The element of claim 46, wherein the stretched polymer has a defect density is less than 1000 per meter of stretched polymer.

53. The element of claim 46, wherein the stretched polymer has a defect density is less than 400 per meter of stretched polymer.

54. The element of claim 46, wherein the stretched polymer has a defect density is less than 200 per meter of stretched polymer.

55. The element of claim 46, wherein the stretched polymer has a defect density is less than 100 per meter of stretched polymer.

56. The element of claim 46, wherein the stretched polymer has a defect density is less than 20 per meter of stretched polymer.

57. The element of claim 46, wherein the stretched polymer is transparent.

58. The element of claim 57, wherein the stretched polymer is stretched such that molecules of the stretched polymer are not uniaxial aligned.

59. The element of claim 46, wherein the stretched polymer is either woven or unwoven.

60. The element of claim 46, wherein the stretched polymer is transparent and contained within a transparent medium that is index matched to an index of the stretched polymer.

61. The element of claim 46, wherein the stretched polymer has a tenacity greater than 90 g/denier.

62. The element of claim 46, wherein the stretched polymer has a tenacity greater than 100 g/denier.

63. The element of claim 46, wherein the stretched polymer has a tenacity greater than 110 g/denier.

64. The element of claim 46, wherein the stretched polymer has a tenacity greater than 120 g/denier.

65. A polymer filament comprising:
a stretched polyethylene polymer having a tenacity greater than 77 g/denier.

66. The filament of claim 65, wherein the stretched polyethylene polymer is transparent.

67. The filament of claim 65, wherein the stretched polyethylene is stretched such that molecules of the stretched polymer are not uniaxial aligned.

68. The filament of claim 65, wherein the stretched polyethylene polymer is part of a woven product.

69. The filament of claim 65, wherein the stretched polyethylene polymer is part of an unwoven product.

70. The filament of claim 65, wherein the unwoven product is a pre-preg product.

71. A polymer filament comprising:
a stretched polyethylene polymer having a high average molecular weight and a tenacity greater than 77 g/denier.

72. A polymer filament comprising:
a stretched polyethylene polymer having a stretch ratio in excess of 20 and has a ratio of tenacity to stretch ratio (t/Rₛ) of greater than 0.16 GPa.

73. A polymer filament comprising:
a stretched polyethylene polymer having a stretch ratio in excess of 40 and has a ratio of tenacity to stretch ratio (t/Rₛ) of greater than 0.10 GPa.

74. The filament of claim 73, wherein the stretched polymer is a stretched polyethylene polymer.

75. A polymer filament comprising:
a stretched polymer having a stretch ratio in excess of 60 and has a ratio of tenacity to stretch ratio (t/Rₛ) of greater than 0.07 GPa.

76. The filament of claim 75, wherein the stretched polymer is a stretched polyethylene polymer.

77. A polymer filament comprising:
a stretched polymer where (t/log₁₀Rₛ)≧32−0.095Rₛ, where t is tenacity in grams/denier and Rₛ is the stretch ratio.

78. The filament of claim 77, wherein the stretched polymer is a stretched polyethylene polymer.

79. A method of testing stretched polymers comprising:
placing a stretched polymer have a refractive index in a fluid having an index of refraction within 0.1 of the refractive index of the stretched polymer; and
observing the stretched polymer with a polarizing microscope in the dark field mode after at least a predetermined amount of time.

80. The method of claim 79, wherein the predetermined time is short.

81. The method of claim 79, wherein the stretched polymer is a stretched polyethylene polymer.

82. A method of manufacturing stretched polymers comprising:
providing raw materials;
mixing the raw materials;
heating the raw materials such that the raw materials become a slurry;
extruding and then cooling the slurry to form xerogel; and
linearly stretching the xerogel to form a stretch polymer.

83. The method of claim 82, wherein the raw materials includes mineral oil and wherein the heating the raw materials occurs for a period of time insufficient to substantially degrade the mineral oil.

84. The method of claim 82, wherein cooling the xerogel is performed in a plurality of stages.

85. The method of claim 84,
wherein each successive stage of the plurality of stages has a temperature lower than a preceding stage of the plurality of stages.

86. The method of claim 82, wherein cooling the xerogel is performed in a single stage having a graduated temperature from an entry point into the single stage to an exit point of the single stage.

87. The method of claim 82, further comprising:
providing an inert gases atmosphere during at least one of:
providing raw materials, mixing the raw materials, heating the raw materials such that the raw materials become a surry, extruding and then cooling the slurry to form xerogel, and stretching the xerogel to form a stretch polymer.

88. The method of claim 82, wherein the stretching the xerogel to form a stretch polymer is performed without bending the xerogel.

89. The method of claim 82, wherein the stretching the xerogel to form a stretch polymer includes at least one heating step and the stretching the xerogel to form a stretch polymer during the at least one heating step is performed without twisting the xerogel.

90. The method of claim 82, wherein the stretching the xerogel to form a stretch polymer is performed without bending the xerogel.

91. The method of claim 82, wherein the stretching the xerogel to form a stretch polymer includes at least one heating step and the stretching the xerogel to form a stretch polymer during the at least one heating step is performed without twisting the xerogel.

92. The method of claim 82, wherein extruding the slurry produces a plurality of filaments which results in the xerogel being a plurality of xerogel filaments, and further comprising separating the plurality of xerogel filaments.

93. The method of claim 82, further comprising placing the stretched polymer into a transparent medium.

94. The method of claim 93, wherein the stretched polymer in combination with the transparent medium is transparent.

95. The method of claim 82, wherein the stretched polymer is transparent.

96. The method of claim 82, wherein the stretched polymer is a stretched polyethylene polymer.

97. The method of claim 96, wherein the stretched polyethylene polymer is transparent.

98. A method of manufacturing stretched polymers comprising:
extruding and then cooling a slurry of polyethylene and mineral oil to form a gel;
removing the mineral oil from the gel to form unstretched polymer; and
at least partially stretching the unstretched polymer substantially without applying non-axial stresses.

99. The method of claim 98, wherein the non-axial stresses are bending the unstretched polymer.

100. The method of claim 98, wherein removing the mineral oil from the gel to form unstretched polymer occurs substantially without applying non-axial stresses.

101. The method of claim 98, wherein the non-axial stresses are bending either the gel or the unstretched polymer.
